(19) United States
(12) Patent Application Publication
(10) Pub. No.: US 2008/0033045 A1
(43) Pub. Date: Feb. 7, 2008

(54) TREATMENT OF PSYCHIATRIC DISORDERS

(75) Inventors: Mark Laughlin, Sunnyvale, CA (US); Kenton Zavitz, Salt Lake City, UT (US); Suzanne Hendrix, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 11/774,692

(22) Filed: Jul. 9, 2007

Related U.S. Application Data
(60) Provisional application No. 60/819,193, filed on Jul. 7, 2006.

(51) Int. Cl. A61K 31/19; A61P 25/00

(52) U.S. Cl. 514/568

(57) ABSTRACT

The invention relates to the treatment of psychiatric disorders.

### CROSS-REFERENCE TO RELATED APPLICATIONS

This application claims priority under 35 U.S.C. §119 (e) to U.S. provisional application Ser. Nos. 60/819,193, filed Jul. 7, 2006.

### FIELD OF THE INVENTION

The invention relates to the treatment and prevention of psychiatric disorders.

### BACKGROUND OF THE INVENTION

Dementia is a brain disorder that seriously affects a person's ability to carry out normal daily activities. Among older people, Alzheimer's disease (AD) is the most common form of dementia and involves parts of the brain that control thought, memory, and language. Despite intensive research throughout the world, the causes of AD are still unknown and there is no cure. AD most commonly begins after the age of 60 with the risk increasing with age. Younger people can also get AD, but it is much less common. It is estimated that 3 percent of men and women ages 65 to 74 have AD. Almost half of those ages 85 and older may have the disease. AD is not a normal part of aging. Alzheimer's disease is a complex disease that can be caused by genetic and environmental factors. In the United States alone, four million adults suffer from Alzheimer's disease (AD). Not only does Alzheimer's disease significantly impact the lives of countless families today, it threatens to become even more of a problem as the baby boom generation matures. The economic burden of AD in the United States is estimated to cost over $100 billion a year and the average lifetime cost per patient is estimated to be $174,000. Unfortunately, there is no cure available for AD. One particularly difficult to deal with AD symptom, for patients and their families, are psychiatric events.

The inventors have discovered that Aβ42 lowering agents, e.g., (R)-2-(2-fluoro-4-biphenylyl)propionic acid (USAN name tarenflurbil), can delay the onset of and reduce the incidence of psychiatric events in Alzheimer's disease patients.

### BRIEF SUMMARY OF THE INVENTION

In one embodiment, the invention relates to delaying the onset of a psychiatric disorder, or one or more symptoms thereof In some aspects of this embodiment, the incidence of psychiatric events/disorders in a population of individuals taking the inventive therapy (e.g., tarenflurbil) is reduced compared to a control population not taking the therapeutic. The method of this embodiment comprises identifying an individual in need of such treatment, and administering to the individual an Aβ42 lowering agent in an amount sufficient to delay the onset of a psychiatric disorder or development of one or more symptoms of a psychiatric disorder. In one aspect of this embodiment, the psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. In one aspect of this embodiment, the Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, or a pharmaceutically acceptable salt thereof. In another aspect of this embodiment, the Aβ42 lowering agent is (R)-2-(2-fluoro-4-biphenylyl)propionic acid, or a pharmaceutically acceptable salt thereof.

In one embodiment, the invention relates to delaying the onset of a psychiatric disorder, or one or more symptoms thereof, in a patient having a neurodegenerative disorder (or that is at risk for developing a neurodegenerative disorder). In some aspects of this embodiment, the incidence of psychiatric events/disorders in a population of individuals taking the inventive therapy (e.g., tarenflurbil) is reduced compared to a control population not taking the therapeutic. The method of this embodiment comprises identifying an individual in need of such treatment, and administering to the individual an Aβ42 lowering agent in an amount sufficient to delay the onset or develop of one or more symptoms of a psychiatric disorder. In one aspect of this embodiment, the psychiatric disorder or symptom thereof is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. In one aspect of this embodiment, the Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, or a pharmaceutically acceptable salt thereof. In some aspects of this embodiment, the individual has a disease or condition chosen from Parkinson's disease, Huntington's disease Alzheimer's disease, Mild cognitive impairment, mild Alzheimer's disease, mild-to-moderate Alzheimer's disease, moderate-to-severe Alzheimer's disease, and dementia.

In one embodiment, the invention relates to treating a psychiatric disorder or one or more symptoms thereof. The method of this embodiment comprises identifying an individual in need of such treatment, and administering to the individual an amount of an Aβ42 lowering agent in an amount sufficient to treat a psychiatric disorder, or one or more symptoms of a psychiatric disorder. In some aspects of this embodiment, the incidence of psychiatric events/disorders in a population of individuals taking the inventive therapy (e.g., tarenflurbil) is reduced compared to a control population not taking the therapeutic. In one aspect of this embodiment, the psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. In one aspect of this embodiment, the Aβ42 lowering agent is chosen from (R)-2-(2-fluoro-4-biphenylyl)propionic acid, 5[1-(2-Fluoro-biphenyl-4-yl)- 1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, or a pharmaceutically acceptable salt thereof.

In some aspects of these embodiments, the psychiatric disorder or symptom thereof is chosen from abnormal behavior, abnormal dreams, aggression, agitation, anger, anxiety, apathy, confusional state, delusion, hallucination, visual hallucination, insomnia, increased libido, mood alterations, mood swings, nightmare, paranoia, psychotic disorder, and sleep disorder.

In some aspects of these embodiments, the psychiatric disorder or symptom thereof is chosen from anger, anxiety, confusional state, delusion, depression, mood swings, nightmare, paranoia, and psychotic disorder. In more specific aspects of these embodiments, the psychiatric disorder or symptom thereof is chosen from anxiety, confusional state, delusion, depression, mood swings, nightmare, paranoia, and psychotic disorder. In even more specific aspects of these embodiments, the psychiatric disorder or symptom thereof is chosen from anxiety, confusional state, delusion, mood swings, nightmare, paranoia, and psychotic disorder.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

In general, the invention relates to a pharmaceutical composition having an Aβ42 lowering agent as the active ingredient, which is useful for preventing, treating and delaying the onset of psychiatric disorders. In one specific embodiment, the invention relates to specific dosage formulations or doses (i.e., unit dosage forms) of (R)-2-(2-fluoro-4-biphenylyl)propionic acid, or a pharmaceutically acceptable salt thereof, useful in the treatment or prevention of psychiatric disorders (and symptoms thereof) in patients with neurodegenerative disorders (e.g., Alzheimer's disease), e.g., 400 mg, 800 mg, 1200 mg, 1600 mg, 2000 mg, 2400 mg compositions or daily doses. As described in more detail below, when the dosage for, for example, the 400 mg dosage form, is orally administered in a single dose of the composition of the invention to a fasting subject under steady state dosing conditions, it provides a Cmax (maximum plasma concentration after administration) of about 30-200 micrograms (μg) per milliliter (mL). When the composition is administered twice daily (b.i.d) for at least 4 months, preferably at least 8 months, and more desirably at least 1 year, it reduces the likelihood of developing a psychiatric disorder, delays the onset of psychiatric disorders, and or reduces the incidence of psychiatric disorders in a population. Such psychiatric disorders include, but are not limited to, abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. The compositions of the invention are formulated with one or more pharmaceutically acceptable excipients, salts, or carriers. The pharmaceutical compositions of the invention can be delivered orally, preferably in a tablet or capsule dosage form. The compositions of the invention can be used in methods for treating, preventing, and prophylaxis against psychiatric disorders in neurodegenerative disorders such as Alzheimer's disease, Parkinson's disease, and Huntington's disease.

In one aspect, the invention provides a dosage comprising (R)-2-(2-fluoro-4-biphenylyl)propionic acid, or a pharmaceutically acceptable salt thereof, in an amount of about 200 mg to about 1200 mg per dose. Oral administration of a single dose to a fasting subject under constant dosing conditions (steady state), provides a Cmax of about 30-500 μg per mL, about 30-400 μg per mL, about 30-300 μg per mL, about 30-200 μg per mL. Oral administration of the composition of this aspect of the invention twice daily (b.i.d) for at least 4 months, preferably at least 8 months, and more desirably at least 1 year, treats, prevent, and/or delays the onset of a psychiatric disorder (or one or more symptoms thereof).

Desirably, the oral dose is provided in capsule or tablet form. In a specific embodiment of this aspect of the invention, the dosage is provided as a pharmaceutical composition composed of (R)-2-(2-fluoro-4-biphenylyl)propionic acid, or a pharmaceutically acceptable salt, a release agent, and optionally additional ingredients. In another specific embodiment of this aspect of the invention, the dosage is provided as a pharmaceutical composition in a unit dosage form that is a tablet composed of (R)-2-(2-fluoro-4-biphenylyl)propionic acid, or a pharmaceutically acceptable salt thereof, microcrystalline cellulose, colloidal silicon dioxide, and magnesium stearate. In another specific embodiment of this aspect of the invention, the dosage is provided as a pharmaceutical composition in a unit dosage form that is a coated tablet composed of (R)-2-(2-fluoro-4-biphenylyl)propionic acid or a pharmaceutically acceptable salt thereof, microcrystalline cellulose, colloidal silicon dioxide, and magnesium stearate, all coated in a mixture of lactose monohydrate, hydroxyl propyl methyl cellulose, titanium dioxide, tracetin/glycerol triacetate, and iron oxide. In another specific embodiment of this aspect of the invention, the dosage is provided as a pharmaceutical composition in a unit dosage form that is a capsule composed of (R)-2-(2-fluoro-4-biphenylyl)propionic acid or a pharmaceutically acceptable salt thereof, microcrystalline cellulose, colloidal silicon dioxide, and magnesium stearate.

In a related aspect, the invention provides for a method of treating or delaying the onset of a psychiatric disorder in an individual having, or suspected of having, Alzheimer's disease, comprising administering (R)-2-(2-fluoro-4-biphenylyl)propionic acid, wherein said administration of a single dose under steady state dosing conditions provides a Cmax of about 30 to about 500 μg per mL. In a more specific embodiment, said Cmax is between 30 and 400 μg per mL. In a more specific embodiment, said Cmax is between 30 and 300 μg per mL. In a more specific embodiment, said Cmax is between 30 and 200 μg per mL. In a more specific embodiment, said Cmax is between 40 and 150 μg per mL.

Psychiatric disorders generally include Adjustment Disorders, Anxiety Disorders, Dissociative Disorders, Eating Disorders, Impulse-Control Disorders, Mood Disorders, Sexual Disorders, Sleep Disorders, Psychotic Disorders, Sexual Dysfunctions, Somatoform Disorders, Substance Disorders, and Personality Disorders. See the DSM IV for information pertaining to the identification and diagnosis of psychiatric disorders as well as other neurodegenerative disorders including Alzheimer's disease. The DSM IV is expressly incorporated by reference in its entirety (Diagnostic and Statistical Manual of Mental Disorders—Fourth Edition (DSM-IV), published by the American Psychiatric Association, Washington D.C., 1994).

Specific psychiatric disorders include Acute Stress Disorder, Adjustment Disorder, Adjustment Disorder with Anxiety, Adjustment Disorder with Depressed Mood, Adjustment Disorder with Disturbance of Conduct, Adjustment Disorder with Mixed Anxiety and Depressed Mood, Adjustment Disorder with Mixed Disturbance of Emotions and Conduct, Agoraphobia without History of Panic Disorder Anxiety Disorders, Anorexia Nervosa Eating Disorders, Antisocial Personality Disorder, Personality Disorders, Anxiety Disorder Due to Medical Condition, Anxiety Disorder NOS, Avoidant Personality Disorder, Bipolar Disorder NOS, Bipolar I Disorder Most Recent Episode Depressed (in full remission), Bipolar I Disorder Most Recent Episode Depressed (in partial remission), Bipolar I Disorder most recent episode depressed (mild), Bipolar I Disorder Most Recent Episode Depressed (Moderate), Bipolar I Disorder most recent episode depressed (severe with psychotic features), Bipolar I Disorder, most recent episode depressed (severe without psychotic features), Bipolar I Disorder most recent episode depressed (unspecified), Bipolar I Disorder most recent episode manic (in full remission), Bipolar I Disorder most recent episode manic (in partial remission), Bipolar I Disorder most recent episode manic (mild), Bipolar I Disorder most recent episode manic (moderate), Bipolar I Disorder most recent episode manic (severe with psychotic features), Bipolar I Disorder most recent episode manic (severe without psychotic features), Bipolar I Disorder most recent episode manic (unspecified), Bipolar I Disorder most recent episode mixed (in full remission), Bipolar I Disorder, most recent episode mixed in partial remission, Bipolar I Disorder most recent episode mixed (mild), Bipolar I Disorder most recent episode mixed (moderate), Bipolar I Disorder most recent episode mixed (severe with psychotic features), Bipolar I Disorder most recent episode mixed severe without psychotic features, Bipolar I Disorder most recent episode mixed (unspecified), Bipolar I Disorder most recent episode unspecified, Bipolar I Disorder most recent episode hypomanic, Bipolar I Disorder single manic episode in full remission, Bipolar I Disorder single manic episode in partial remission, Bipolar I Disorder single manic episode (mild), Bipolar I Disorder single manic episode (moderate), Bipolar I Disorder single manic episode (severe with psychotic features), Bipolar I Disorder single manic episode severe without psychotic features, Bipolar I Disorder single manic episode (unspecified), Bipolar II Disorder, Body Dysmorphic Disorder,Borderline Personality Disorder, Breathing-Related Sleep Disorder, Brief Psychotic Disorder, Bulimia Nervosa, Circadian Rhythm Sleep Disorder, Conversion Disorder, Cyclothymic Disorder, Delusional Disorder, Dependent personality Disorder, Depersonalization disorder, Depressive Disorder NOS, Dissociative Amnesia, Dissociative Disorder NOS, Dissociative fugue, Dissociative Identity Disorder, Dissociative Disorders, Dyspareunia, Dyssomnia NOS, Dyssomnia Related to (Another Disorder), Dysthymic Disorder, Eating disorder NOS, Exhibitionism, Female Dyspareunia Due to Medical Condition, Female Hypoactive Sexual Desire Disorder Due to Medical Condition, Female Orgasmic Disorder, Female Sexual Arousal Disorder, Fetishism Sexual Disorders, Frotteurism Sexual Disorders, Gender Identity Disorder in Adolescents or Adults, Gender Identity Disorder in Children, Gender Identity Disorder NOS, Generalized Anxiety Disorder, Histrionic Personality Disorder, Hypoactive Sexual Desire Disorder, Hypochondriasis, Impulse—Control Disorder NOS, Insomnia Related to Another Disorder, Intermittent Explosive Disorder, Kleptomania, Major Depressive Disorder Recurrent (in full remission), Major Depressive Disorder Recurrent (in partial remission), Major Depressive Disorder Recurrent (Mild), Major Depressive Disorder recurrent (moderate), Major Depressive Disorder recurrent (severe with psychotic features), Major Depressive Disorder, Recurrent (severe without psychotic features), Major Depressive Disorder Recurrent (unspecified), Major Depressive Disorder Single Episode (in full remission), Major Depressive Disorder single episode (in partial remission), Major Depressive Disorder single episode (Mild), Major Depressive Disorder single episode, Major Depressive Disorder single episode (severe with psychotic features), Major Depressive Disorder single episode (severe without psychotic features), Major Depressive Disorder single episode (unspecified), Male Dyspareunia Due to Medical Condition, Male Erectile Disorder, Male Erectile Disorder Due to Medical Condition, Male Hypoactive Sexual Desire Disorder Due to Medical Condition, Male Orgasmic Disorder, Mood Disorder Due to Medical Condition, Narcissistic Personality Disorder, Narcolepsy Sleep Disorders, Nightmare Disorder Sleep Disorders, Obsessive Compulsive Disorder, Obsessive-Compulsive Personality Disorder, Other Female Sexual Dysfunction Due to Medical Condition, Other Male Sexual Dysfunction Due to Medical Condition, Pain Disorder Associated with both Psychological Factors and Medical Conditions, Pain Disorder Associated with Psychological Features, Panic Disorder with Agoraphobia, Panic Disorder without Agoraphobia, Paranoid Personality Disorder, Paraphilia NOS, Parasomnia NOS, Pathological Gambling, Pedophilia Sexual Disorders, Personality Disorder NOS, Posttraumatic Stress Disorder, Premature Ejaculation, Primary Hypersomnia, Primary Insomnia, Psychotic Disorder Due to Medical Condition with Delusions, Psychotic Disorder Due to Medical Condition with Hallucinations, Psychotic Disorder NOS, Pyromania, Schizoaffective Disorder, Schizoid Personality Disorder, Schizophrenia Catatonic Type, Schizophrenia Disorganized Type, Schizophrenia Paranoid Type, Schizophrenia Residual Type, Schizophrenia Undifferentiated Type, Schizophreniform Disorder, Schizotypal Personality Disorder, Sexual Aversion Disorder, Sexual Disorder NOS, Sexual Dysfunction NOS, Sexual Masochism, Sexual Sadism, Shared Psychotic Disorder, Sleep Disorder Due to A Medical Condition Hypersomnia Type, Sleep Disorder Due to A Medical Condition Insomnia Type, Sleep Disorder Due to A Medical Condition Mixed Type, Sleep Disorder Due to A Medical Condition Parasomnia Type, Sleep Terror Disorder, Sleepwalking Disorder, Social Phobia, Somatization Disorder, Somatoform Disorder NOS, Specific Phobia, Trichotillomania, Undifferentiated Somatoform Disorder, Vaginismus Sexual Disorders, and Voyeurism.

In one embodiment, the invention relates to delaying the onset of a psychiatric disorder, or one or more symptoms thereof. The method of this embodiment comprises identifying an individual in need of such treatment, and administering to the individual an Aβ42 lowering agent in an amount sufficient to delay the onset of a psychiatric disorder or development of one or more symptoms of a psychiatric disorder. In one aspect of this embodiment, the psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. In one aspect of this embodiment, the Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, or a pharmaceutically acceptable salt thereof. In another aspect of this embodiment, the Aβ42 lowering agent is (R)-2-(2-fluoro-4-biphenylyl)propionic acid, or a pharmaceutically acceptable salt thereof. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is from about 50 to about 3000 milligrams per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 800 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1000 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1200 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1400 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1600 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1800 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 2000 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1600 milligrams per day (e.g., 800 mg twice daily). In one aspect of this embodiment, the patient is administered a second therapeutic agent (e.g., an acetylcholine esterase inhibitor). In some aspects of this embodiment, the incidence of psychiatric events in the treated population is reduced compared to similar “control” population that has not been treated with the Aβ42 lowering agent (e.g., (R)-2-(2-fluoro-4-biphenylyl)propionic acid).

In one embodiment, the invention relates to delaying the onset of a psychiatric disorder, or one or more symptoms thereof, in a patient having a neurodegenerative disorder (or that is at risk for developing a neurodegenerative disorder). The method of this embodiment comprises identifying an individual in need of such treatment, and administering to the individual an Aβ42 lowering agent in an amount sufficient to delay the onset or develop of one or more symptoms of a psychiatric disorder. In one aspect of this embodiment, the psychiatric disorder or symptom thereof is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. In one aspect of this embodiment, the Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, or a pharmaceutically acceptable salt thereof. In another aspect of this embodiment, the Aβ42 lowering agent is (R)-2-(2-fluoro-4-biphenylyl)propionic acid, or a pharmaceutically acceptable salt thereof. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is from about 50 to about 3000 milligrams per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 800 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1000 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1200 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1400 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1600 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1800 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 2000 milligrams or more per day. In one aspect of this embodiment, the amount of (R)-2-(2-fluoro-4-biphenylyl)propionic acid, or a pharmaceutically acceptable salt thereof, sufficient to delay the onset of a psychiatric disorder, or one or more symptoms thereof, is about 1600 milligrams per day (e.g., 800 mg twice daily). In one aspect of this embodiment, the patient having a neurodegenerative disorder has mild-to-moderate Alzheimer's disease, mild Alzheimer's disease, or mild cognitive impairment. In one aspect of this embodiment, the patients has mild Alzheimer's disease. The skilled artisan can readily identify such patients using clinical assessments. In one aspect of this embodiment, the patient has a ratio of Aβ42/Aβ40 that is declining in plasma or CSF. In one aspect of this embodiment, the patient has an elevated plasma Aβ42/Aβ40 ratio and/or high plasma Aβ42 levels. In some aspects of this embodiment, the incidence of psychiatric events in the treated population is reduced compared to similar “control” population that has not been treated with the Aβ42 lowering agent (e.g., (R)-2-(2-fluoro-4-biphenylyl)propionic acid).

In one embodiment, the invention relates to treating a psychiatric disorder, or one or more symptoms thereof. The method of this embodiment comprises identifying an individual in need of such treatment, and administering to the individual an amount of an Aβ42 lowering agent in an amount sufficient to treat a psychiatric disorder, or one or more symptoms of a psychiatric disorder. In one aspect of this embodiment, the psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder. In one aspect of this embodiment, the Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, or a pharmaceutically acceptable salt thereof. In one aspect of this embodiment, the amount of (R)-2-(2-fluoro-4-biphenylyl)propionic acid, or a pharmaceutically acceptable salt thereof, sufficient to treat the psychiatric disorder, or one or more symptoms thereof, is from about 50 to about 3000 milligrams per day. In one aspect of this embodiment, the amount of (R)-2-(2-fluoro-4-biphenylyl)propionic acid, or a pharmaceutically acceptable salt thereof, sufficient to treat the psychiatric disorder, or one or more symptoms thereof, is about 800 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof, is about 1000 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof, is about 1200 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof, is about 1400 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof, is about 1600 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof, is about 1800 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof, is about 2000 milligrams or more per day. In one aspect of this embodiment, the amount of (R)-2-(2-fluoro-4-biphenylyl)propionic acid, or a pharmaceutically acceptable salt thereof, sufficient to treat a psychiatric disorder, or one or more symptoms thereof is about 1600 milligrams per day (e.g., 800 mg twice daily). In some aspects of this embodiment, the incidence of psychiatric events in the treated population is reduced compared to similar “control” population that has not been treated with the Aβ42 lowering agent (e.g., (R)-2-(2-fluoro-4-biphenylyl)propionic acid).

In some aspects of these embodiments, the psychiatric disorder or symptom thereof is chosen from abnormal behavior, abnormal dreams, aggression, agitation, anger, anxiety, apathy, confusional state, delusion, hallucination, visual hallucination, insomnia, increased libido, mood alterations, mood swings, nightmare, paranoia, psychotic disorder, and sleep disorder.

In some aspects of these embodiments, the psychiatric disorder or symptom thereof is chosen from anger, anxiety, confusional state, delusion, depression, mood swings, nightmare, paranoia, and psychotic disorder.

Aβ42 Lowering Agents

The Aβ42 lowering agents for use in the invention can be a known Aβ42 lowering agents such as (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, or 2-(2-fluoro-1,1′-biphenyl-4-yl)-2-methylpropionic acid. Examples of Aβ42 lowering agents for use in the combination formulations and treatments of the invention are given in, e.g., WO 01/78721, WO 2004/073705, WO 2004/064771, WO 2004/074232, and PCT/US05/09595 (each of which is herein incorporated by reference).

Aβ42 lowering agents include, but are not limited to, those having the following Formulae:

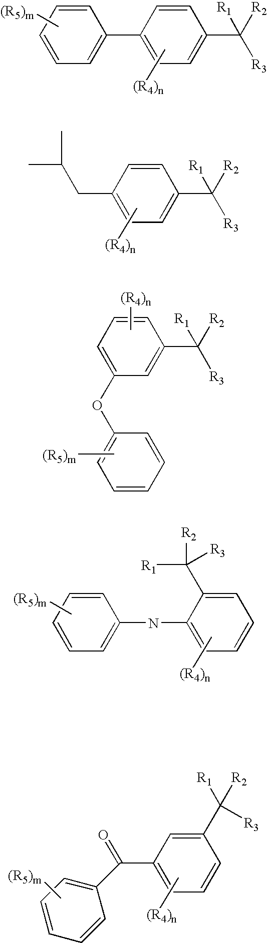

Where R₁ is chosen from —CH₃, —CH₂CH₃, —CH₂CH₂CH₃, and —CH₂CH₂CH₂CH₃ (or can be taken together with R₂ to give a cyclopropyl ring, a cyclobutyl ring, a cyclopentyl ring, or a cyclohexyl ring);

R₂ is chosen from —CH₃, —CH₂CH₃, —CH₂CH₂CH₃, and —CH₂CH₂CH₂CH₃, (or can be taken together with R₂ to give a cyclopropyl ring, a cyclobutyl ring, a cyclopentyl ring, or a cyclohexyl ring);

R₃ is chosen from —COOH, —COOR₆, —CONH₂, —CONHR₆, —CONR₆R₇, —CONHSO₂R₆, tetrazolyl, and a —COOH bioisostere;

R₄ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₅ is chosen from —Cl, —F, —Br, —I, —CF₃, —OCF₃, —SCF₃, —OCH₃, —OCH₂CH₃, —CN, —CH═CH₂, —CH₂OH, and —NO₂;

R₆ is chosen from —CH₃, —CH₂CH₃, —CH₂CH₂CH₃, and —CH₂CH₂CH₂CH₃.

R₇ is chosen from —CH₃, —CH₂CH₃, —CH₂CH₂CH₃, and —CH₂CH₂CH₂CH₃.

M is an integer chosen from 0, 1, 2, and 3.

N is an integer chosen from 0, 1, 2, and 3.

Examples of compounds (i.e., the one or more second compounds) for use in the invention include those as shown above (and those listed below), including enantiomers, diastereomers, racemates, and pharmaceutically acceptable salts thereof. The compounds described in this invention disclosure can be made by an ordinary artisan skilled in the art of organic chemistry synthesis.

Additional Aβ42 lowering agents for use in the invention include, but are not limited to, 2-methyl-2 (2-fluoro-4′-trifluoromethylbiphen-4-yl)propionic acid; 2-methyl-2(2-fluoro-4′cyclohexylbiphen-4-yl)propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro -4′-hydroxybiphenyl-4-yl)cyclopropanecarboxylic acid;1-[2-fluoro-4′-(tetrahydropyran-4-yloxy) biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl) benzoyl]amino][1,1′-biphenyl]-4-yl]-cyclopropanecarboxylic acid; 1-[2-fluoro-4′-[[4-(trifluoromethyl) cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid;1-(2,3′-difluoro-4″-hydroxy[1,1′:4′,1″-tert-phenyl]-4-yl)-cyclopropane-carboxylic acid; 1-(2,2′-difluoro-4″-hydroxy [1,1′:4′,1″-tert-phenyl]-4-yl)-cyclopropane-carboxylic acid; 2-(2-fluoro-3′,5′-bis(chloro)biphen-4-yl) propionic acid amide; 2-(2-fluoro-4′-trifluoromethylbiphen-4-yl)propionic acid; 2-(2-fluoro-3′-trifluoromethylbiphen-4-yl) propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl) -2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole.

Aβ₄₂ lowering agents can be identified by a number of methods. To identify Aβ₄₂ lowering agents that reduce APP processing, a biological composition having an APP processing activity (i.e. an activity that processes APP into various Aβ forms, one of which is Aβ₄₂), is incubated with APP under conditions in which APP processing occurs. To identify Aβ₄₂ lowering agents that increase Aβ₄₂ catabolism, a biological composition having Aβ₄₂ catabolic activity is incubated with Aβ₄₂ under conditions in which Aβ₄₂ catabolism occurs. Depending on the nature of the biological composition, the APP or Aβ₄₂ substrate can be added to the biological composition, or, each or both can be a component of the biological composition. APP processing or Aβ₄₂ catabolism is allowed to take place in the presence or absence of the candidate Aβ₄₂ lowering agent. The level of Aβ₄₂ generated from APP processing or the level of Aβ₄₂ remaining after the catabolic reaction, in the presence and absence of the candidate Aβ₄₂ lowering agent, is determined and compared. Aβ₄₂ lowering agents useful for treating AD are those that reduce the level of Aβ₄₂ either by reducing APP processing into Aβ₄₂ or by enhancing Aβ₄₂ catabolism and increasing Aβ₃₈ production. The biological composition having an APP processing and/or catabolic activity can be a cell-free biological sample. For example, a cell-free biological sample can be a purified or partially purified enzyme preparation; it also can be a cell lysate generated from cells able to process APP into Aβ₄₂ or from cells able to catabolize Aβ₄₂. Cell lysates can be prepared using known methods such as, for example, sonication or detergent-based lysis. In the case of an enzyme preparation or cell lysate, APP can be added to the biological composition having the APP processing activity, or Aβ₄₂ can be added to the biological composition having Aβ₄₂ catabolic activity.

In addition, the biological composition can be any mammalian cell that has an APP processing activity as well as a nucleic acid vector encoding APP. Alternatively, the biological composition can be any mammalian cell that has Aβ catabolic activity as well as a nucleic acid vector or a viral nucleic acid-based vector containing a gene that encodes Aβ₄₂. The vector typically is an autonomously replicating molecule, a molecule that does not replicate but is transiently transfected into the mammalian cell, or a vector that is integrated into the genome of the cell. Typically, the mammalian cell is any cell that can be used for heterologous expression of the vector-encoded APP or Aβ₄₂ in tissue culture. For example, the mammalian cell can be a Chinese hamster ovary (CHO) cell, a fibroblast cell, or a human neuroglioma cell. The mammalian cell also can be one that naturally produces APP and processes it into Aβ₄₂, or one that naturally produces and catabolizes Aβ₄₂.

Further, the biological composition can be an animal such as a transgenic mouse that is engineered to over-express a form of APP that then is processed into Aβ₄₂. Alternatively, the animal can be a transgenic mouse that is engineered to over-express Aβ₄₂. Animals can be, for example, rodents such as mice, rats, hamsters, and gerbils. Animals also can be rabbits, dogs, cats, pigs, and non-human primates, for example, monkeys.

To perform an in vitro cell-free assay, a cell-free biological sample having an activity that can process APP into Aβ₄₂ is incubated with the substrate APP under conditions in which APP is processed into various Aβ forms including Aβ₄₂ (see Mclendon et al. (2000) FASEB 14:2383-2386). Alternatively, a cell-free biological sample having an activity that can catabolize Aβ₄₂ is incubated with the substrate Aβ₄₂ under conditions in which Aβ₄₂ is catabolized. To determine whether a candidate Aβ₄₂ lowering agent has an effect on the processing of APP into Aβ₄₂ or the catabolism of Aβ₄₂, two reactions are compared. In one reaction, the candidate Aβ₄₂ lowering agent is included in the processing or catabolic reaction, while in a second reaction, the candidate Aβ₄₂ lowering agent is not included in the processing or catabolic reaction. Levels of the different Aβ forms produced in the reaction containing the candidate Aβ₄₂ lowering agent are compared with levels of the different Aβ forms produced in the reaction that does not contain the candidate Aβ₄₂ lowering agent.

The different Aβ forms can be detected using any standard antibody based assays such as, for example, immunoprecipitation, western hybridization, and sandwich enzyme-linked immunosorbent assays (ELISA). Different Aβ forms also can be detected by mass spectrometry; see, for example, Wang et al. (1996) J Biol Chem 271:31894-902. Levels of Aβ species can be quantified using known methods. For example, internal standards can be used as well as calibration curves generated by performing the assay with known amounts of standards.

In vitro cell-based assays can be used determine whether a candidate Aβ₄₂ lowering agent has an effect on the processing of APP into Aβ₄₂ or an effect on catabolism of Aβ₄₂. Typically, cell cultures are treated with a candidate Aβ₄₂ lowering agent. Then the level of Aβ₄₂ in cultures treated with a candidate Aβ₄₂ lowering agent is compared with the level of Aβ₄₂ in untreated cultures. For example, mammalian cells expressing APP are incubated under conditions that allow for APP expression and processing as well as Aβ₄₂ secretion into the cell supernatant. The level of Aβ₄₂ in this culture is compared with the level of Aβ₄₂ in a similarly incubated culture that has been treated with the candidate Aβ₄₂ lowering agent. Alternatively, mammalian cells expressing Aβ₄₂ are incubated under conditions that allow for Aβ₄₂ catabolism. The level of Aβ₄₂ in this culture is compared with the level of Aβ₄₂ in a similar culture that has been treated with the candidate Aβ₄₂ lowering agent.

In vivo animal studies also can be used to identify Aβ₄₂ lowering agents useful for treating AD. Typically, animals are treated with a candidate Aβ₄₂ lowering agent and the levels of Aβ₄₂ in plasma, CSF, and/or brain are compared between treated animals and those untreated. The candidate Aβ₄₂ lowering agent can be administered to animals in various ways. For example, the candidate Aβ₄₂ lowering agent can be dissolved in a suitable vehicle and administered directly using a medicine dropper or by injection. The candidate Aβ₄₂ lowering agent also can be administered as a component of drinking water or feed. Levels of Aβ in plasma, cerebral spinal fluid (CSF), and brain are determined using known methods. For example, levels of Aβ₄₂ can be determined using sandwich ELISA or mass spectrometry in combination with internal standards or a calibration curve. Plasma and CSF can be obtained from an animal using standard methods. For example, plasma can be obtained from blood by centrifugation, CSF can be isolated using standard methods, and brain tissue can be obtained from sacrificed animals.

When present in an in vitro or in vivo APP processing or Aβ₄₂ catabolic reaction, Aβ₄₂ lowering agents reduce the level of Aβ₄₂ generated by APP processing or remaining following Aβ catabolism. For example, in an in vitro cell-free assay, the level of Aβ₄₂ is reduced due to either a reduction of APP processing or an increase in Aβ₄₂ catabolism in the presence the Aβ₄₂ lowering agent. In an in vitro cell culture study, a reduction in the level of Aβ₄₂ secreted into the supernatant results from the effect of the Aβ₄₂ lowering agent on either a reduction in processing of APP into Aβ₄₂ or an increased catabolism of Aβ₄₂. Similarly, in animal studies, a reduction in the level of Aβ₄₂ that can be detected in plasma, CSF, or brain is attributed to the effect of the Aβ₄₂ lowering agent on either a reduction in the processing of APP into Aβ₄₂ or an increase in the catabolism of Aβ₄₂.

The level of Aβ₄₂ can be reduced by a detectable amount. For example, treatment with an Aβ₄₂ lowering agent leads to a 0.5, 1, 3, 5, 7, 15, 20, 40, 50, or more than 50% reduction in the level of Aβ₄₂ generated by APP processing or remaining following Aβ₄₂ catabolism when compared with that in the absence of the Aβ₄₂ lowering agent. Preferably, treatment with the Aβ₄₂ lowering agent leads to at least a 20% reduction in the level of Aβ₄₂ generated when compared to that in the absence of Aβ₄₂ lowering agent. More preferably, treatment with an Aβ₄₂ lowering agent leads to at least a 40% reduction the level of Aβ₄₂ when compared to that in the absence of an Aβ₄₂ lowering agent.

Examples of Aβ42 lowering agents for use in the formulations and treatments of the invention are given in, e.g., WO 01/78721, WO 2004/073705, WO 2004/064771, and WO 2004/074232 (each of which is herein incorporated by reference).

Dosages, formulations, and route of administration

The active compounds of this invention are typically administered in combination with a pharmaceutically acceptable carrier through any appropriate routes such as parenteral, oral, or topical administration, in a therapeutically (or prophylactically) effective amount according to the methods set forth above. A preferred route of administration for use in the invention is oral administration.

Generally, the toxicity profile and therapeutic efficacy of the therapeutic agents can be determined by standard pharmaceutical procedures in suitable cell models or animal models. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but remains significantly below the LD₅₀ dosage level, as determined from cell or animal models.

Typically, the compounds and compositions for use in the invention can be effective at an amount of from about 0.05 mg to about 4000 mg per day, preferably from about 0.1 mg to about 2000 mg per day. However, the amount can vary with the body weight of the patient treated and the state of disease conditions. The active ingredient may be administered at once, or may be divided into a number of smaller doses to be administered at predetermined intervals of time.

In the case of combination therapy, a therapeutically effective amount of another therapeutic compound can be administered in a separate pharmaceutical composition, or alternatively included in the pharmaceutical composition according to the present invention. The pharmacology and toxicology of other therapeutic compositions are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in the art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Soft gelatin capsules can be prepared in which capsules contain a mixture of the active ingredient and vegetable oil or non-aqueous, water miscible materials such as, for example, polyethylene glycol and the like. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, or gelatin.

Tablets for oral use are typically prepared in the following manner, although other techniques may be employed. The solid substances are ground or sieved to a desired particle size, and the binding agent is homogenized and suspended in a suitable solvent. The active ingredient and auxiliary agents are mixed with the binding agent solution. The resulting mixture is moistened to form a uniform suspension. The moistening typically causes the particles to aggregate slightly, and the resulting mass is gently pressed through a stainless steel sieve having a desired size. The layers of the mixture are then dried in controlled drying units for determined length of time to achieve a desired particle size and consistency. The granules of the dried mixture are gently sieved to remove any powder. To this mixture, disintegrating, anti-friction, and anti-adhesive agents are added. Finally, the mixture is pressed into tablets using a machine with the appropriate punches and dies to obtain the desired tablet size. The operating parameters of the machine may be selected by the skilled artisan.

If the compound for use in the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound for use in the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium. These substituents may optionally be further substituted with a substituent selected from such groups.

The formulations and unit dosage forms of the invention can have a number of different ingredients. Depending on the dosage strength, a unit dosage form has an amount of active pharmaceutical ingredient(s) (API) sufficient for achieving a therapeutic effect in a target population. Additionally “inactive pharmaceutical ingredients” need to be present to achieve a therapeutically effect release of the API. Thus the amount and type of inactive ingredients help achieve a therapeutically effective release of the therapeutic agent. In one aspect of the invention, a tablet unit dosage form is provided having the following inactive ingredients: one or more disintegrants in an amount sufficient to facilitate break-up (disintegration) of the tablet after administration (e.g., provide an immediate release dissolution profile), one or more binders in an amount sufficient to impart adequate cohesiveness to the tablet and/or provide adequate free flowing qualities by formulation of granules of desired size/hardness, one or more diluents in an amount sufficient to impart satisfactory compression characteristics, one or more lubricants in an amount sufficient to provide an adequate flow rate of the granulation and/or prevent adhesion of the material to the die/punch, reduce interparticle friction, and/or facilitate ejection from the die, and if desired, optional ingredients.

The disintegration rate, and often the dissolution rate of a compacted solid pharmaceutical formulation in an aqueous environment (e.g., the patient's stomach) may be increased by the addition of a disintegrant to the formulation. Disintegrants include alginic acid, carboxymethylcellulose calcium, carboxymethylcellulose sodium (e.g., Ac-Di-Sol® Primellose®.), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®) and starch.

Solid pharmaceutical formulations that are compacted into a dosage form, such as a tablet, may include excipients whose functions include helping to bind the active pharmaceutical ingredient and other excipients together after compression. Binders for solid pharmaceutical formulations include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methylcellulose (e.g. Methocel®), lactose, liquid glucose, magnesium aluminum silicate, maltodextrin, methylcellulose, polymethacrylates, povidone (e.g. Kollidon®, Plasdone®), pregelatinized starch, sodium alginate and starch. Glidants can be added to improve the flowability of a non-compacted solid formulation and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered formulation, the formulation is subjected to pressure from a punch and dye. Some excipients and active pharmaceutical ingredients have a tendency to adhere to the surfaces of the punch and dye, which can cause the product to have pitting and other surface irregularities. A lubricant can be added to the formulation to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Examples of diluents include, but are not limited to, calcium carbonate, calcium phosphate, calcium sulfate, cellulose, cellulose acetate, compressible sugar, confectioner's sugar, dextrates, dextrin, dextrose, ethyl cellulose, fructose, fumaric acid, glyceryl palmitostearate, hydrogenated vegetable oil, kaolin, lactitol, lactose, magnesium carbonate, magnesium oxide, maltodextrin, maltose, mannitol, medium chaim glyceride, microcrystalline cellulose, polydextrose, polymethylacrylates, simethicone, sodium alginate, sodium chloride, sorbitol, starch, pregelantized starch, sterilizable maize, sucrose, sugar spheres, talc, tragacanth, trehalose, and xylitol.

Examples of disintegrants include, but are not limited to, alginic acid, calcium phosphate, carboxymethyl cellulose calcium, carboxymethyl cellulose sodium, powdered cellulose, chitosan, crospovidone, docusate sodium, guar gum, hydroxylpropyl cellulose, magnesium aluminum silicate, methylcellulose, poidone, sodium alginate, sodium starch glycolate, starch, and pregelantinized starch.

Example of binders (binding agents) include, but are not limited to, acacia, alginic acid, carbomers, carboxymethyl cellulose sodium, carrageenan, cellulose acetate phthalate, ceratonia, chitosan, confectioners sugar, cottonseed oil, dextrates, dextrin, dextrose, ethylcellulose, gelatin, glucose, glyceryl behenate, guar gum, hydrogenated vegetable oil, hydroxyethyl cellulose, hydroxyethylmethyl cellulose, hydroxylpropyl cellulose, hypromellose, magnesium aluminum silicate, maltodextrin, maltodextrin, maltose, methylcellulose, microcrystalline cellulose, poloxamer, polydextrose, polyethylene oxide, polymethyl acrylates, povidone, sodium alginate, starch, pregelantized starch, stearic acid, sucrose, sunflower oil, and zein.

Examples of lubricants include, but are not limited to, calcium stearate, glycerin monostearate, glyceryl behenate, glyceryl palmitostearate, hydrogenated castor oil, hydrogenated vegetable oil, light mineral oil, magnesium lauryl sulfate, magnesium stearate, medium chain triglycerides, mineral oil, poloxamer, polyethylene glycol, sodium benzoate, sodium chloride, sodium lauryl sulfate, sodium stearyl fumarate, stearic acid, talc, and zinc stearate.

Examples of glidants include, but are not limited to, calcium phosphate, calcium silicate, cellulose powdered, colloidal silicon dioxide, magnesium silicate, magnesium trisilicate, silicon dioxide, starch, and talc.

Optional ingredients in the formulations of the invention include, but are not limited to, flavors, coloring agents, and stabilizers.

Flavoring agents and flavor enhancers make the dosage form more palatable to the patient. Common flavoring agents and flavor enhancers for pharmaceutical products that may be included in the formulation of the present invention include maltol, vanillin, ethyl vanillin, menthol, citric acid, fumaric acid, ethyl maltol and tartaric acid.

Solid and liquid formulations may also be dyed using any pharmaceutically acceptable colorant to improve their appearance and/or facilitate patient identification of the product and unit dosage level.

In one embodiment, the tablet unit dosage form has a hardness of about 5 kp (kilopond) or more, about 7 kp or more, about 9 kp or more, about 11 kp or more, and about 13 kp or more to avoid excessive friability, and a hardness of about 20 kp or less, about 19 kp or less, about 18 kp or less, about 17 kp or less, and about 16 kp or less, is desirable to avoid subsequent difficulty in hydrating the tablet when exposed to gastric fluid. In some aspects of this embodiment, the hardness of the tablet unit dosage form is from 9 kp to 18 kp, 11 kp to 17 kp, and 13 kp to 17 kp. When hardness is in an acceptable range, tablet friability is typically less than about 1.0%, preferably less than about 0.8% and more preferably less than about 0.5%, in a standard test. Some issues that may cause variations in tablet hardness are inconsistent tablet weight, particle size variations, poor powder compressibility, and insufficient binder level.

The tablet unit dosage forms of the invention have a friability of less than about 1%, less than about 0.9%, less than about 0.8%, less than about 0.7%, less than about 0.6%, less than about 0.5%, and less than about 0.4% (all at 100 rev).

### EXAMPLE

A clinical trial with (R)-2-(2-fluoro-4-biphenylyl)propionic acid (USAN name of tarneflurbil) was conducted according to the method disclosed in U.S. patent application Ser. No. 10/889,971, filed Jul. 12, 2004, (US publication no. 20050042284) which is expressly incorporated herein by reference in its entirety. In this clinical trial, Alzheimer's disease patients were treated with placebo, 400 mg tarenflurbil twice daily, or 800 mg tarenflurbil twice daily for one year. Analysis of the psychiatric disorder events yielded the following results.

| | Placebo | | 400 mg BID | | 800 mg BID | | Total | |
|---|---|---|---|---|---|---|---|---|
| Psychiatric Disorders (MedDRA) | (N = 50) | | (N = 40) | | (N = 50) | | (N = 140) | |
| Total Number of Adverse Events | 21 | | 17 | | 10 | | 48 | |
| Number of Patients With at Least | 18 | (36%) | 12 | (30%) | 7 | (14%) | 37 | (26.4%) |
| One Adverse Event (Psychiatric Disorder) | | | | | | | | |
| Abnormal behaviour | 1 | (2%) | 0 | | 0 | | 1 | (0.7%) |
| Abnormal dreams | 1 | (2%) | 0 | | 0 | | 1 | (0.7%) |
| Aggression | 2 | (4%) | 0 | | 0 | | 2 | (1.4%) |
| Agitation | 3 | (6%) | 2 | (5%) | 1 | (2%) | 6 | (4.3%) |
| Anger | 0 | | 1 | (2.5%) | 0 | | 1 | (0.7%) |
| Anxiety | 2 | (4%) | 2 | (5%) | 2 | (4%) | 6 | (4.3%) |
| Apathy | 1 | (2%) | 0 | | 0 | | 1 | (0.7%) |
| Confusional state | 2 | (4%) | 4 | (10%) | 0 | | 6 | (4.3%) |
| Delusion | 0 | | 1 | (2.5%) | 0 | | 1 | (0.7%) |
| Depression | 2 | (4%) | 1 | (2.5%) | 4 | (8%) | 7 | (5.0%) |
| Hallucination | 1 | (2%) | 0 | | 0 | | 1 | (0.7%) |
| Hallucination, visual | 1 | (2%) | 1 | (2.5%) | 0 | | 2 | (1.4%) |
| Insomnia | 1 | (2%) | 0 | | 1 | (2%) | 2 | (1.4%) |
| Libi do increased | 1 | (2%) | 1 | (2.5%) | 0 | | 2 | (1.4%) |
| Mood altered | 0 | | 0 | | 1 | (2%) | 1 | (0.7%) |
| Mood swings | 0 | | 1 | (2.5%) | 0 | | 1 | (0.7%) |
| Nightmare | 1 | (2%) | 0 | | 1 | (2%) | 2 | (1.4%) |
| Paranoia | 0 | | 1 | (2.5%) | 0 | | 1 | (0.7%) |
| Psychotic disorder | 0 | | 1 | (2.5%) | 0 | | 1 | (0.7%) |
| Sleep disorder | 1 | (2%) | 1 | (2.5%) | 0 | | 2 | (1.4%) |

Statistical analysis revealed a significant reduction (p=0.02) in the number of psychiatric events in the treatment group and a delay in time to progression to psychiatric events (p=0011).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method of treating or delaying the onset of a psychiatric disorder in an individual, comprising administering to said individual a therapeutically effective amount of an Aβ42 lowering agent, or a pharmaceutically acceptable salt thereof.

2. The method of claim 1, wherein said Aβ42 lowering agent is chosen from 2-methyl-2 (2-fluoro-4′-trifluoromethylbiphen-4-yl)propionic acid; 2-methyl-2 (2-fluoro-4′cyclohexylbiphen-4-yl)propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro-4′-hydroxybiphenyl-4-yl)cyclopropanecarboxylic acid; 1-[2-fluoro-4′-(tetrahydropyran-4-yloxy)biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4, 4-dimethylcyclohexyl)-2-fluoro[1,1′-biphenyl]-4-yl]-cyclopropane-carboxylic acid; 1-[2-fluoro-4′-[[4-(trifluoromethyl) benzoyl]amino][1,1′-biphenyl]-4-yl]-cyclopropanecarboxylic acid; 1-[2-fluoro-4′-[[4-(trifluoromethyl) cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]- cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1″-tert-phenyl]-4-yl)-cyclopropane-carboxylic acid; 1-(2,2′-difluoro-4″-hydroxy[1,1′: 4′,1″-tert-phenyl]-4-yl)-cyclopropane-carboxylic acid; 2-(2-fluoro-3′,5′-bis(chloro)biphen-4-yl)propionic acid amide; 2-(2-fluoro-4′-trifluoromethylbiphen-4-yl)propionic acid; 2-(2-fluoro-3′-trifluoromethylbiphen-4-yl) propionic acid; 2-(2-fluoro-3′,5′-bis(trifluoromethyl)biphen-4-yl)propionic acid; 2-(4′-cyclohexyl-2-fluorobiphen-4-yl) propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole, and pharmaceutically acceptable salts thereof.

3. The method of claim 1, wherein said Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, and pharmaceutically acceptable salts thereof.

4. The method of claim 1, wherein said Aβ42 lowering agent is (R)-2-(2-fluoro-4-biphenylyl)propionic acid or a pharmaceutically acceptable salt thereof.

5. The method of claim 4, wherein about 800 mg of (R)-2-(2-fluoro-4-biphenylyl)propionic acid, or a bioequivalent amount of a pharmaceutically acceptable salt thereof, is administered to said individual, twice daily.

6. The method of claim 1, wherein said psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder.

7. A method of treating or delaying the onset of a psychiatric disorder in a patient having a neurodegenerative disorder comprising administering to said patient a therapeutically effective amount of an Aβ42 lowering agent, or a pharmaceutically acceptable salt thereof.

8. The method of claim 7, wherein said Aβ42 lowering agent is chosen from 2-methyl-2(2-fluoro-4′-trifluoromethylbiphen-4-yl) propionic acid; 2-methyl-2 (2-fluoro-4′cyclohexyl biphen-4-yl)propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro -4′-hydroxybiphenyl-4-yl)cyclopropanecarboxylic acid; 1-[2-fluoro-4′-(tetrahydropyran-4-yloxy) biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4,4-dimethylcyclohexyl)-2-fluoro [1,1′-biphenyl]-4-yl]-cyclopropane- carboxylic acid; 1-[2-fluoro-4′-[[4-(trifluoromethyl) benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]- cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1″-tert-phenyl]-4-yl)-cyclopropane-carboxylic acid; 1-(2,2′-difluoro-4″-hydroxy[1,1′:4′,1″-tert-phenyl]-4-yl)-cyclopropane-carboxylic acid; 2-(2-fluoro-3′,5′-bis(chloro)biphen-4-yl)propionic acid amide; 2-(2-fluoro-4′-trifluoromethylbiphen-4-yl)propionic acid; 2-(2-fluoro-3′-trifluoromethylbiphen-4-yl)propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl) -2-methylpropanoic acid; 2-Methyl-2-(3-phenoxy-phenyl)-propionic acid; 2-(4-Isobutyl-phenyl)-2-methyl-propionic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole, and pharmaceutically acceptable salts thereof.

9. The method of claim 7, wherein said Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, and pharmaceutically acceptable salts thereof.

10. The method of claim 7, wherein said Aβ42 lowering agent is (R)-2-(2-fluoro-4-biphenylyl)propionic acid or a pharmaceutically acceptable salt thereof.

11. The method of claim 10, wherein about 800 mg of (R)-2-(2-fluoro-4-biphenylyl)propionic acid, or a bioequivalent amount of a pharmaceutically acceptable salt thereof, is administered twice daily.

12. The method of claim 7, wherein said psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder.

13. A method of treating or delaying the onset of a psychiatric disorder in a patient having Alzheimer's disease comprising administering to said patient a therapeutically effective amount of an Aβ42 lowering agent, or a pharmaceutically acceptable salt thereof.

14. The method of claim 13, wherein said Aβ42 lowering agent is chosen from 2-methyl-2 (2-fluoro-4′-trifluoromethylbiphen-4-yl)propionic acid; 2-methyl-2 (2-fluoro-4′cyclohexyl biphen-4-yl)propionic acid; 1-(2-fluoro-4′-trifluoromethylbiphenyl-4-yl)cyclopropanecarboxylic acid; 1-(4′-cyclohexyl-2-fluorobiphenyl-4-yl)cyclopropanecarboxylic acid; 1-(4′-benzyloxy-2-fluorobiphenyl-4-yl)cyclopropanecarboxylic acid; 1-(2-fluoro-4′-isopropyloxybiphenyl-4-yl)cyclopropanecarboxylic acid; 1-(2-fluoro-3′-trifluoromethoxybiphenyl-4-yl)cyclopropanecarboxylic acid; 1-(2-fluoro-4′-trifluoromethoxybiphenyl-4-yl)cyclopropanecarboxylic acid; 1-(2-fluoro-3′-trifluoromethylbiphenyl-4-yl)cyclopropanecarboxylic acid; 1-(4′-cyclopentyl-2-fluorobiphenyl-4-yl)cyclopropanecarboxylic acid; 1-(4′-cycloheptyl-2-fluorobiphenyl-4-yl)cyclopropanecarboxylic acid; 1-(2′-cyclohexyl-2-fluorobiphenyl-4-yl)cyclopropanecarboxylic acid; 1-(2-fluoro -4′-hydroxybiphenyl-4-yl)cyclopropanecarboxylic acid; 1-[2-fluoro-4′-(tetrahydropyran-4-yloxy) biphenyl-4-yl]-cyclopropane-carboxylic acid; 1-(2,3′,4′-trifluorobiphenyl-4-yl)cyclopropanecarboxylic acid; 1-(3′,4′-dichloro-2-fluorobiphenyl-4-yl)cyclopropanecarboxylic acid; 1-(3′,5′-dichloro-2-fluorobiphenyl-4-yl)cyclopropanecarboxylic acid 1-(3′-chloro-2,4′-difluorobiphenyl-4-yl)cyclopropanecarboxylic acid; 1-(4-benzo[b]thiophen-3-yl-3-fluorophenyl)cyclopropanecarboxylic acid; 1-(2-fluoro-4′-prop-2-inyloxy-biphenyl-4-yl)-cyclopropanecarboxylic acid; 1-(4′-cyclohexyloxy-2-fluoro-biphenyl-4-yl)-cyclopropanecarboxylic acid; 1-[2-fluoro-4′-(tetrahydropyran-4-yl)-biphenyl-4-yl]-cyclopropanecarboxylic acid; 1-[2-fluoro-4′-(4-oxo-cyclohexyl)-biphenyl-4-yl]-cyclopropanecarboxylic acid; 2-(2″-fluoro-4-hydroxy-[1,1′:4′,1″]tert-phenyl-4″-yl)-cyclopropanecarboxylic acid; 1-[4′-(4,4-dimethylcyclohexyl)-2-fluoro[1,1′-biphenyl]-4-yl]-cyclopropane-carboxylic acid; 1-[2-fluoro-4′-[[4-(trifluoromethyl) benzoyl]amino][1,1′-biphenyl]-4-yl]-cyclopropanecarboxylic acid; 1-[2-fluoro-4′-[[4-(trifluoromethyl)cyclohexyl]oxy][1,1′-biphenyl]-4-yl]-cyclopropanecarboxylic acid; 1-[2-fluoro-4′-[(3,3,5,5-tetramethylcyclohexyl)oxy][1,1′-biphenyl]-4-yl]-cyclopropanecarboxylic acid; 1-[4′-[(4,4-dimethylcyclohexyl)oxy]-2-fluoro[1,1′-biphenyl]-4-yl]-cyclopropanecarboxylic acid; 1-(2,3′,4″-trifluoro[1,1′:4′,1″-tert-phenyl]-4-yl)-cyclopropanecarboxylic acid; 1-(2,2′,4″-trifluoro[1,1′:4′,1″-tert-phenyl]-4-yl)-cyclopropanecarboxylic acid; 1-(2,3′-difluoro-4″-hydroxy[1,1′:4′,1″-tert-phenyl]-4-yl)-cyclopropane-carboxylic acid; 1-(2,2′-difluoro-4″-hydroxy[1,1′:4′,1″-tert-phenyl]-4-yl)-cyclopropane-carboxylic acid; 2-(2-fluoro-3′,5′-bis(chloro)biphen-4-yl)propionic acid amide; 2-(2-fluoro-4′-trifluoromethylbiphen-4-yl)propionic acid; 2-(2-fluoro-3′-trifluoromethylbiphen-4-yl)propionic acid; 2-(2-fluoro-3′,5′-bis(trifluoromethyl)biphen-4-yl)propionic acid; 2-(4′-cyclohexyl-2-fluorobiphen-4-yl)propionic acid; 2-(2-Fluoro-1,1′-biphenyl-4-yl)-2-methylpropanoic acid; 2-Methyl-2-(3-phenoxy-phenyl)-propionic acid; 2-(4-Isobutyl-phenyl)-2-methyl-propanoic acid; 2-(6-Chloro-9H-carbazol-2-yl)-2-methyl-propionic acid; 2-[1-(4-Chloro-benzoyl)-5-methoxy-2-methyl-1H-indol-3-yl]-2-methyl-propionic acid; and 5-[1-(2-Fluoro-biphenyl-4-yl)-1-methyl-ethyl]-2H-tetrazole, and pharmaceutically acceptable salts thereof.

15. The method of claim 13 wherein said Aβ42 lowering agent is chosen from (R)-2-(2-fluoro-4-biphenylyl)propionic acid, 5[1-(2-Fluoro-biphenyl-4-yl)-1-methyl-ethyl]-2H-tetrazole, 2-(4-isobutyl-phenyl)-2-methyl propionic acid, and 2-(2-fluoro-1,1′-biphenyl-4-yl)-2-methylpropionic acid, and pharmaceutically acceptable salts thereof.

16. The method of claim 15, wherein said Aβ42 lowering agent is (R)-2-(2-fluoro-4-biphenylyl)propionic acid or a pharmaceutically acceptable salt thereof.

17. The method of claim 16, wherein about 800 mg of (R)-2-(2-fluoro-4-biphenylyl)propionic acid, or a bioequivalent amount of a pharmaceutically acceptable salt thereof, is administered twice daily.

18. The method of claim 13, wherein said psychiatric disorder, or symptom thereof, is chosen from abnormal behavior, abnormal dreams, aggression, agitation, anger, anxiety, apathy, confusional state, delusion, depression, hallucination, visual hallucination, insomnia, increased libido, mood alterations, mood swings, nightmare, paranoia, psychotic disorder, and sleep disorder.
